(19) United States
(12) Patent Application Publication
(10) Pub. No.: US 2012/0071718 A1
(43) Pub. Date: Mar. 22, 2012

(54) ENDOSCOPE APPARATUS AND METHOD OF CONTROLLING ENDOSCOPE APPARATUS

(75) Inventor: Seigo ON, Tokyo (JP)

(73) Assignee: OLYMPUS CORPORATION, Tokyo (JP)

(21) Appl. No.: 13/235,833

(22) Filed: Sep. 19, 2011

(30) Foreign Application Priority Data
Sep. 21, 2010 (JP) 2010-210469

(51) Int. Cl. A61B 1/04

(52) U.S. Cl. 600/109

(57) ABSTRACT

An endoscope apparatus includes an observation magnification acquisition section that acquires magnification state information that indicates a state of an observation magnification, a motion detection section that detects motion amount information that indicates a relative motion amount of an object and an imaging section of the endoscope apparatus, an imaging period control section that reduces an imaging period of each image that is indicated by an image signal captured by the endoscope apparatus based on the magnification state information and the motion amount information, and a light source control section that controls intensity of illumination light applied to the object corresponding to the imaging period.

Japanese Patent Application No. 2010-210469 filed on Sep. 21, 2010, is hereby incorporated by reference in its entirety.

### BACKGROUND

The present invention relates to an endoscope apparatus, a method of controlling an endoscope apparatus, and the like.

In recent years, an endoscope apparatus including an objective optical system that can implement normal observation and magnifying observation has been widely used in order to improve the diagnostic accuracy when performing a medical examination on an internal organ inside the body cavity. Specifically, the user specifies an area that is likely to be a lesion area during normal observation. The user moves the end of the endoscope apparatus closer to the specified area, observes the specified area in detail in a magnifying observation mode, and determines a treatment measure.

However, when observing the specified area in a state in which the end of the endoscope apparatus is positioned close to the specified area (i.e., magnifying observation), the effects of the movement of the endoscope apparatus, the movement of the object, and the like increase as compared with those during normal observation, so that it may be difficult to accurately examine the specified area. JP-A-8-46859 discloses a method that intermittently changes the shutter speed of the imaging element of the endoscope apparatus in units of a given number of fields, and adjusts the gain of the image signal corresponding to the shutter speed.

### SUMMARY

According to one aspect of the invention, there is provided an endoscope apparatus comprising:

an observation magnification acquisition section that acquires magnification state information that indicates a state of an observation magnification;

a motion detection section that detects motion amount information that indicates a relative motion amount of an object and an imaging section of the endoscope apparatus;

an imaging period control section that reduces an imaging period of each image that is indicated by an image signal captured by the endoscope apparatus based on the magnification state information and the motion amount information; and

a light source control section that controls intensity of illumination light applied to the object corresponding to the imaging period.

According to another aspect of the invention, there is provided a method of controlling an endoscope apparatus comprising:

acquiring magnification state information and motion amount information, the magnification state information indicating a state of an observation magnification, and the motion amount information indicating a relative motion amount of an object and an imaging section of the endoscope apparatus;

reducing an imaging period of each image that is indicated by an image signal captured by the endoscope apparatus based on the acquired magnification state information and the acquired motion amount information; and

controlling intensity of illumination light applied to the object corresponding to the reduced imaging period.

### BRIEF DESCRIPTION OF THE DRAWINGS

FIG. 1 shows a configuration example of an endoscope apparatus according to one embodiment of the invention.

FIG. 2 is a view illustrative of an exposure time (shutter speed) during normal observation.

FIG. 3 is a view illustrative of an exposure time (shutter speed) during magnifying observation.

FIG. 4 shows a configuration example of a light source section.

FIG. 5 shows an example in which an imaging period (exposure time) is instantaneously reduced.

FIG. 6 shows an example in which an imaging period (exposure time) is gradually reduced.

FIG. 7 shows an example of a data structure stored in a ROM.

FIG. 8 shows another configuration example of an endoscope apparatus.

FIG. 9 is a view illustrative of a frame rate during normal observation.

FIG. 10 is a view illustrative of a frame rate during magnifying observation.

FIG. 11 shows an example in which an imaging period is reduced by reducing a frame rate.

FIG. 12 shows another configuration example of an endoscope apparatus.

FIG. 13 shows a configuration example of an AGC section.

FIGS. 14A and 14C show examples of a smoothing filter used during magnifying observation, and FIGS. 14B and 14D show examples of a smoothing filter used during normal observation.

FIG. 15 shows another configuration example of an endoscope apparatus.

FIG. 16 shows yet another configuration example of an endoscope apparatus.

FIG. 17 is a view illustrative of an illumination period during normal observation.

FIG. 18 is a view illustrative of an illumination period during magnifying observation.

FIG. 19 shows an example in which an imaging period (illumination period) is instantaneously reduced.

FIG. 20 shows an example in which an imaging period (illumination period) is gradually reduced.

### DESCRIPTION OF EXEMPLARY EMBODIMENTS

Several aspects of the invention may provide an endoscope apparatus, a method of controlling an endoscope apparatus, and the like that can acquire a bright image while suppressing blurring by reducing the imaging period while increasing the intensity of illumination light emitted from a light source during magnifying observation.

Several aspects of the invention may provide an endoscope apparatus, a method of controlling an endoscope apparatus, and the like that can suppress blurring during magnifying observation while suppressing an increase in the amount of noise contained in the image signal and maintaining the brightness of the image signal by controlling the intensity of illumination light emitted from a light source while increasing the shutter speed or the frame rate, or reducing the illumination period using light emitted from the light source.

According to one embodiment of the invention, there is provided an endoscope apparatus comprising:

an observation magnification acquisition section that acquires magnification state information that indicates a state of an observation magnification;

a motion detection section that detects motion amount information that indicates a relative motion amount of an object and an imaging section of the endoscope apparatus;

an imaging period control section that reduces an imaging period of each image that is indicated by an image signal captured by the endoscope apparatus based on the magnification state information and the motion amount information; and

a light source control section that controls intensity of illumination light applied to the object corresponding to the imaging period.

According to the above embodiment, the magnification state information is acquired, and the imaging period is controlled based on the acquired magnification state information. The intensity of light is also controlled while controlling the imaging period. Therefore, the effects of blurring can be reduced by controlling the imaging period, and a situation in which the image darkens can be prevented by controlling the intensity of illumination light while controlling the imaging period.

According to another embodiment of the invention, there is provided a method of controlling an endoscope apparatus comprising:

acquiring magnification state information and motion amount information, the magnification state information indicating a state of an observation magnification, and the motion amount information indicating a relative motion amount of an object and an imaging section of the endoscope apparatus;

reducing an imaging period of each image that is indicated by an image signal captured by the endoscope apparatus based on the acquired magnification state information and the acquired motion amount information; and

controlling intensity of illumination light applied to the object corresponding to the reduced imaging period.

Exemplary embodiments of the invention are described below. Note that the following exemplary embodiments do not in any way limit the scope of the invention laid out in the claims. Note also that all of the elements described in connection with the following exemplary embodiments should not necessarily be taken as essential elements of the invention.

### 1. Method

An endoscope apparatus used in recent years generally includes an optical system that implements normal observation and magnifying observation (i.e., observation that utilizes a magnification higher than that of normal observation) in order to accurately observe an in vivo object. For example, the object can be observed at a magnification equal to or higher than 100 during magnifying observation.

However, the effects of blurring may increase during magnifying observation due to a high magnification. The term “blurring” includes blurring caused by the movement of the imaging section (insertion section) of the endoscope apparatus due to an operation performed by the user (doctor), for example. Since the object is an in vivo tissue, the object is not necessarily stationary (e.g., the esophagus located near the heart may be affected by pulsation). Therefore, blurring may occur due to the movement of the object.

It is necessary to reduce the effects of blurring during magnifying observation so that the doctor can smoothly perform diagnosis, treatment, and the like. The shutter speed of the imaging section may be adjusted in order to reduce the effects of blurring, for example. Specifically, the effects of blurring may be reduced by increasing the shutter speed (e.g., reducing the charge storage time of the imaging element when utilizing an electronic shutter). However, since the brightness of the acquired image signal decreases due to a decrease in exposure time, the doctor may not be able to smoothly perform diagnosis, treatment, and the like using the endoscope apparatus.

The present application proposes a method that reduces the imaging period of each image (video image) that is indicated by an image signal captured by the imaging section, and controls the intensity of illumination light in synchronization with the reduction in imaging period in order to reduce the effects of blurring within one frame.

Specifically, the imaging period (e.g., the charge storage time or the exposure period of the imaging element) is reduced during magnifying observation while increasing the intensity of illumination light. For example, the intensity of illumination light is doubled when the imaging period is halved. This makes it possible to reduce the effects of blurring within one frame, and maintain the brightness of the acquired image signal at a level equal to that when the imaging period is not reduced.

Note that the imaging element and illumination are enabled in the imaging period when acquiring (capturing) a single image, for example. The expression “the imaging element is enabled” used herein means that photodiodes are operated so that a photoelectric conversion process is performed (i.e., an electronic shutter is opened), for example. The expression “illumination is enabled” used herein means that illumination light is emitted at an intensity sufficient for the imaging element to detect light and generate image information. The imaging period may be reduced by reducing the frame rate (second embodiment), disabling the imaging element (first embodiment), or disabling illumination (fifth embodiment), for example.

A first embodiment illustrates a method that controls the imaging period by controlling the charge storage time (shutter speed) of the imaging element, and a second embodiment illustrates a method that controls the imaging period by controlling the frame rate.

A third embodiment illustrates a method that performs a gain-up process when a sufficiently bright image signal cannot be acquired even if the intensity of illumination light is maximized.

In the first to third embodiments, a blurring correction process is performed during magnifying observation (e.g., when the observation magnification is equal to or higher than a given threshold value). A fourth embodiment illustrates a case where the process is performed taking account of the motion amount. Specifically, the blurring correction process is performed when the observation state (mode) is magnifying observation, and the relative motion amount of the object and the imaging section is equal to or larger than a given threshold value.

A fifth embodiment illustrates a case where the imaging period is controlled by controlling the illumination period using a light source (e.g., LED light source) that can be blinked.

### 2. First Embodiment

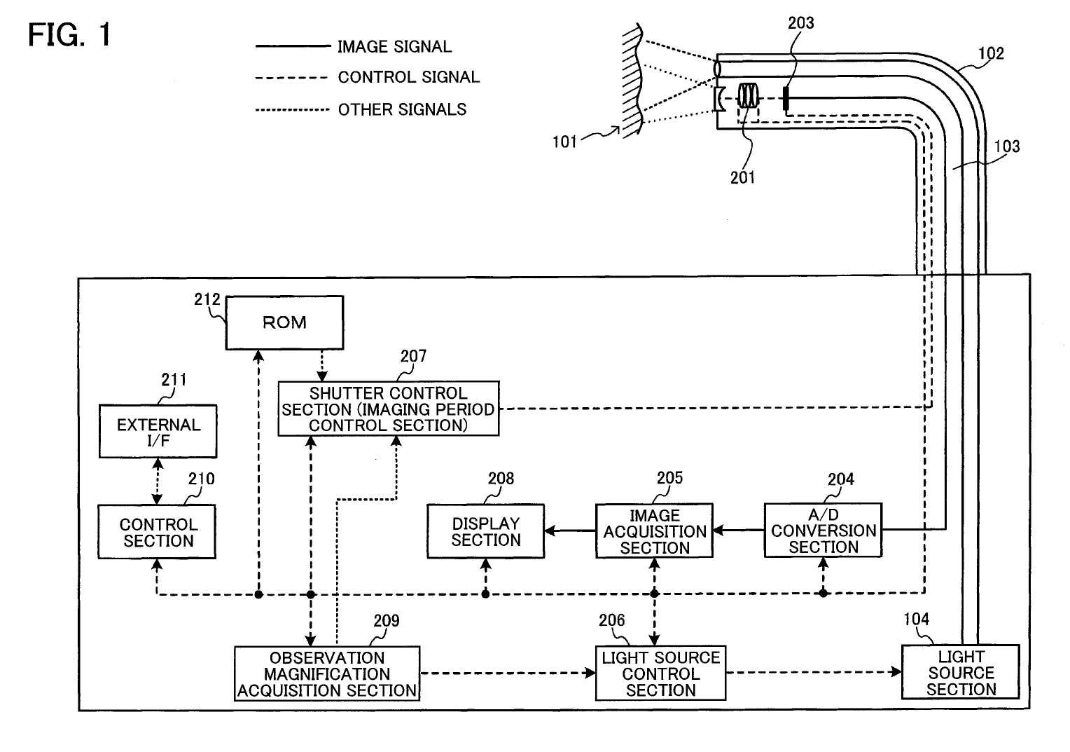

FIG. 1 shows a configuration example of an endoscope apparatus according to the first embodiment. The endoscope apparatus that observes an object 101 includes an insertion section 102, a light guide 103, a light source section 104, a lens system 201, an imaging element 203, an A/D conversion section 204, an image acquisition section 205, a light source control section 206, a shutter control section 207, a display section 208, an observation magnification acquisition section 209, a control section 210, an external OF 211, and a ROM 212.

The insertion section 102 of the endoscope apparatus has an elongated shape and can be curved so that the insertion section 102 can be inserted into a body cavity (body). Light emitted from the light source section 104 is applied to the object 101 via the light guide 103 that can be curved. The lens system 201 is disposed on the end of the insertion section 102. Reflected light from the object 101 enters the imaging element 203 via the lens system 201. The imaging element 203 includes a Bayer color filter array. An analog image signal generated (converted) by the imaging element 203 is transmitted to the A/D conversion section 204.

The A/D conversion section 204 is connected to the display section 208 via the image acquisition section 205. The observation magnification acquisition section 209 is connected to the light source control section 206 and the shutter control section 207. The light source control section 206 is connected to the light source section 104. The shutter control section 207 is connected to the imaging element 203. The ROM 212 is connected to the shutter control section 207. The light source section 104 is connected to the rear end of the light guide 103. Light emitted from the light source section 104 reaches the end of the insertion section 102 via the light guide 103, and is applied to the object 101. The control section 210 is bidirectionally connected to the lens system 201, the A/D conversion section 204, the image acquisition section 205, the light source control section 206, the shutter control section 207, the display section 208, the observation magnification acquisition section 209, the external I/F 211, and the ROM 212.

The A/D conversion section 204 converts the analog image signal output from the imaging element 203 into a digital image signal (hereinafter referred to as “image signal”), and transmits the image signal to the image acquisition section 205. The image acquisition section 205 performs image processing on the image signal input from the A/D conversion section 204 under control of the control section 210. Specifically, the image acquisition section 205 performs a Bayer interpolation process (i.e., a process that converts a Bayer image signal into an RGB image signal), a white balance process, a color management process, a grayscale transformation process, and the like. The image acquisition section 205 transmits the resulting image signal (RGB signal) to the display section 208, and the display section 208 displays the image.

The endoscope apparatus according to the first embodiment is configured so that the object is observed while changing the observation mode (state) between normal observation and magnifying observation (i.e., changing the observation magnification of the observation optical system). When the user (doctor) has found a lesion area during a medical examination, the user moves the end of the endoscope apparatus closer to the lesion area, increases the observation magnification (changes the observation mode to magnifying observation), and observes the lesion area. When the user has determined that the lesion area is malignant, the user treats the lesion area by endoscopic submucosal dissection (ESD) or the like. When the user cannot determine whether or not the lesion area is malignant, the user collects a sample of the lesion area, performs a pathological examination on the sample, and determines a treatment strategy. The endoscope apparatus according to the first embodiment is configured so that the endoscope apparatus can be controlled by the user via the external I/F 211, and the control section 210 adjusts the observation magnification by controlling the lens system 201 so that the observation mode is changed between normal observation and magnifying observation. The observation mode is determined to be magnifying observation when the observation magnification acquired by the observation magnification acquisition section 209 is higher than a given observation magnification threshold value, and is determined to be normal observation when the observation magnification acquired by the observation magnification acquisition section 209 is lower than the given observation magnification threshold value. Note that the observation mode (normal observation or magnifying observation) need not necessarily be determined based on the observation magnification.

A lesion area can be accurately determined (diagnosed) by magnifying observation as compared with normal observation. However, the displayed image may be blurred to a large extent during magnifying observation as compared with normal observation due to peristalsis in an internal organ inside the body cavity, an operation performed by the user, and the like. Moreover, the effects of blurring may increase as the magnification increases.

The endoscope apparatus according to the first embodiment is configured so that blurring of the image during magnifying observation is suppressed by increasing the shutter speed, and the brightness of the image signal is maintained by increasing the intensity of light emitted from the light source.

Specifically, the endoscope apparatus performs the following process. When the user has moved the end of the endoscope apparatus closer to the lesion area, the control section 210 adjusts the lens system 201 based on operation information input from the external I/F 211 so that the observation mode is changed from normal observation to magnifying observation. The observation magnification acquisition section 209 acquires information that changes the observation mode to magnifying observation or normal observation (e.g., observation magnification information) from the control section 210, and causes the light source control section 206 and the shutter control section 207 to operate. The shutter control section 207 adjusts the imaging period by controlling an electronic shutter of the imaging element.

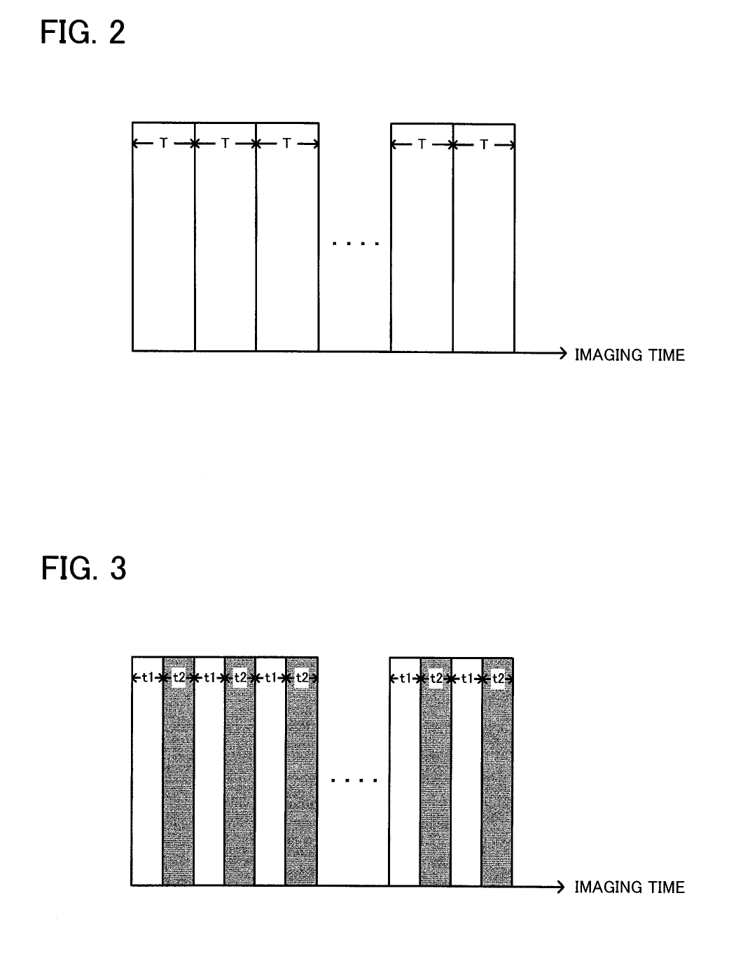

As shown in FIG. 2, the exposure time during normal observation is set to a given time T in time series, and the imaging element 203 sequentially converts the reflected light from the object 101 into an analog image signal. As shown in FIG. 3, when the observation mode is changed from normal observation to magnifying observation, the exposure time is set to a given time t1 within the time T by controlling the electronic shutter of the imaging element 203, and the imaging element 203 converts the reflected light from the object 101 into an analog image signal. An image is not captured within a given time t2 subsequent to the time t1. The relationship between the times t1, t2, and T is shown by the following expression (1). The information about the times t1, t2, and T may be stored in the ROM 212 in advance, or the user may input the information about the times t1, t2, and T via the external I/F 211. Note that the imaging time (imaging period) (exposure time) is returned to the time T when the observation mode is changed from magnifying observation to normal observation.

T=t1+t2 (1)

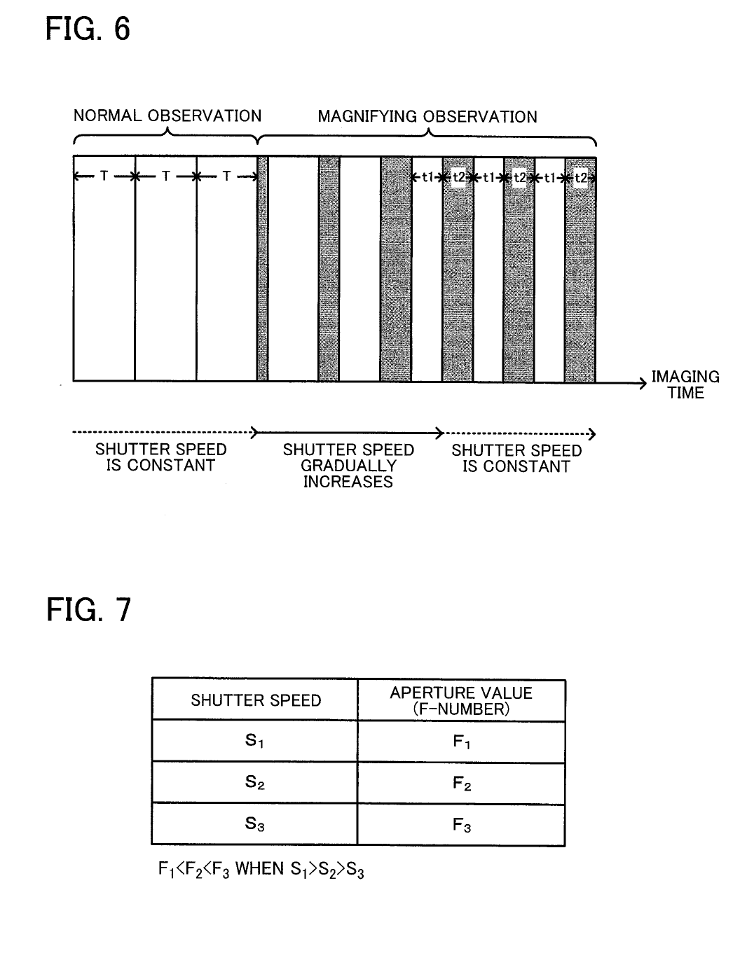

When the observation mode is changed from normal observation to magnifying observation, the imaging time may be instantaneously changed from the time T to the time t1 (see FIG. 5), or may be gradually reduced from the time T to the time t1 (see FIG. 6). When the observation mode is changed from magnifying observation to normal observation, the imaging time may be instantaneously changed from the time t1 to the time T, or may be gradually increased from the time t1 to the time T.

Since the exposure time is reduced using the electronic shutter during magnifying observation while maintaining the same imaging frame rate as that employed during normal observation, blurring of the image can be suppressed. However, the brightness of the image signal acquired during magnifying observation decreases as compared with the brightness of the image signal acquired during normal observation due to a decrease in exposure time.

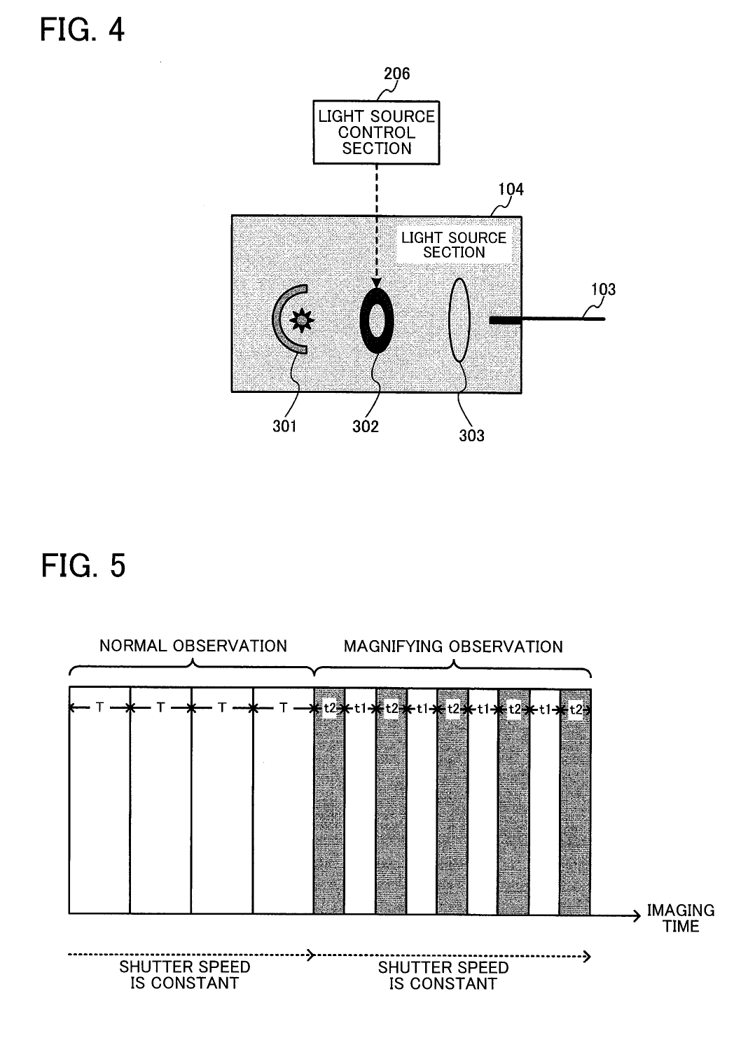

The endoscope apparatus according to the first embodiment solves the above problem by controlling the shutter speed and the intensity of light emitted from the light source. FIG. 4 shows an example of the configuration of the light source section 104. The light source section 104 includes a light source 301, a light source aperture 302, and an illumination optical system 303. Light emitted from the light source 301 enters the light guide 103 via the light source aperture 302 and the illumination optical system 303. When the user performs a medical examination using the endoscope apparatus, the user can adjust the intensity of light emitted from the light source in a plurality of stages by controlling the light source aperture 302 via the external I/F 211 corresponding to the medical examination state. Note that the intensity of light emitted from the light source may be automatically adjusted based on the brightness of the image signal. In this case, a plurality of pairs of the shutter speed and the light source aperture value used to adjust the intensity of light emitted from the light source are stored in the ROM 212 in advance. FIG. 7 shows an example of a data structure stored in the ROM 212. Specifically, the F-number decreases (i.e., the aperture increases) as the shutter speed increases (i.e., the exposure time decreases).

When the user changes the observation state (mode), a given light source aperture value is extracted from the ROM 212 corresponding to the shutter speed under control of the control section 210. The light source aperture 302 of the light source section 104 is then adjusted under control of the light source control section 206. For example, the imaging time is adjusted to 1/60th of a second from 1/30th of a second by controlling the electronic shutter of the imaging element, and the intensity of light emitted from the light source is instantaneously doubled corresponding to the shutter speed under control of the light source control section 206. The brightness of the image signal is always kept almost constant by making a fine adjustment using a normal endoscope dimming process.

When the user changes the observation mode to magnifying observation, blurring of the image is suppressed by increasing the shutter speed, and the intensity of light emitted from the light source is adjusted in order to maintain the brightness of the image signal, as described above. For example, the intensity of light applied to the object is increased by opening the light source aperture 302 as compared with that employed during normal observation under control of the light source control section 206. When the user changes the observation mode to normal observation, the light source aperture 302 that has been opened up is adjusted to a value corresponding to the shutter speed employed during normal observation.

When using an LED light source, the intensity of light emitted from the light source can be increased by increasing the amount of current that flows through the light source by increasing the voltage applied to the light source while increasing the shutter speed.

When the user moves the end of the endoscope apparatus closer to the object, and performs magnifying observation, the shutter speed may be gradually increased, and the brightness of the image signal may be maintained using a known dimming process instead of instantaneously increasing the intensity of light emitted from the light source.

The first embodiment has been described above taking a configuration in which the user selects the observation magnification (i.e., normal observation or magnifying observation) via the external I/F 211, and the information about the selected observation magnification is transmitted to the observation magnification acquisition section 209. Note that the configuration is not limited thereto. For example, when the user moves the end of an endoscope apparatus having an autofocus function closer to the object, and observes the object, the position of the lens system 201 may be automatically adjusted so that the object is in focus. In this case, the observation magnification acquisition section 209 acquires information about the in-focus object plane (focal point) under control of the control section 210, and determines the observation state. For example, the observation magnification acquisition section 209 compares the in-focus object plane with a given threshold value, and determines that the observation state is magnifying observation when the in-focus object plane is less (shorter) than the threshold value (e.g., the end of the endoscope apparatus is positioned close to the object). The observation magnification acquisition section 209 determines that the observation state is normal observation when the in-focus object plane is greater (longer) than the threshold value (e.g., the end of the endoscope apparatus is positioned away from the object).

As shown in FIG. 1, the endoscope apparatus according to the first embodiment includes the observation magnification acquisition section 209 that acquires magnification state information, an imaging period control section that reduces the imaging period of each image indicated by an image signal captured by the endoscope apparatus based on the magnification state information (i.e., the shutter control section 207 in the first embodiment), and the light source control section 206 that controls the intensity of illumination light corresponding to the imaging period.

The term “magnification state information” used herein refers to information that indicates the state of the observation magnification. The magnification state information may be the observation magnification, or may be information equivalent to the observation magnification. The magnification state information may be information that indicates the observation mode (i.e., normal observation or magnifying observation) that is determined based on the observation magnification.

This makes it possible to implement an endoscope apparatus that acquires the magnification state information, and reduces the imaging period based on the acquired magnification state information. Therefore, the effects of blurring within one frame can be reduced. Since the intensity of illumination light is controlled while reducing the imaging period, a situation in which the image darkens due to a reduction in the imaging period can be prevented. Specifically, an endoscope apparatus that can acquire a bright image (image information) with a small amount of blurring can be implemented.

The light source control section 206 may increase the intensity of illumination light when the imaging period control section has reduced the imaging period.

This makes it possible to emit a sufficient intensity of light even when the imaging period has been reduced (e.g., the shutter speed has increased (i.e., the exposure time has decreased)), so that a bright image can be acquired. The expression “bright” used herein means that the image is brighter as compared with the case where the imaging period has been reduced without increasing the intensity of illumination light (i.e., the image has a brightness almost equal to that when the imaging period has not been reduced).

The imaging period control section may reduce the imaging period in a magnifying observation mode as compared with a normal observation mode. The light source control section 206 may increase the intensity of illumination light in the magnifying observation mode as compared with that employed in the normal observation mode.

Specifically, the observation mode is classified as the magnifying observation mode or the normal observation mode, and the endoscope apparatus reduces the imaging period, and increases the intensity of illumination light in the magnifying observation mode. This makes it possible to perform a blurring correction process (and a process that eliminates a shortage of the intensity of light due to the blurring correction process) when observing the object at a high magnification (i.e., when observation is significantly affected by blurring).

The observation magnification acquisition section 209 may acquire adjustment information about the observation magnification that has been input by the user as the magnification state information. The imaging period control section may reduce the imaging period based on the acquired magnification state information.

The observation magnification acquisition section 209 can thus acquire the adjustment information about the observation magnification input by the user as the magnification state information. The term “adjustment information about the observation magnification” used herein refers to information that is generated by operating a switch, a button, or the like that is included in the external I/F 211 or the like and is used to change the observation magnification, for example. The adjustment information about the observation magnification may be information that is generated by an operation (e.g., button operation) that sets the observation mode to the magnifying observation mode.

The imaging period control section may reduce the imaging period when it has been determined that the observation magnification indicated by the magnification state information is equal to or higher than a given threshold value.

This makes it possible to determine whether the observation mode is the magnifying observation mode or the normal observation mode (i.e., whether or not to perform the blurring correction) by comparing the acquired observation magnification with the given observation magnification threshold value when the observation magnification has been acquired as the magnification state information.

The imaging period control section may include the shutter control section 207 that adjusts the shutter speed corresponding to the magnification state information (see FIG. 1), and may control the imaging period corresponding to the shutter speed adjusted by the shutter control section 207. For example, the shutter control section 207 may adjust the shutter speed based on the exposure time of the imaging element.

Therefore, the imaging period control section can control the imaging period based on the shutter speed (e.g., the charge storage time/exposure time of the imaging element when using an electronic shutter).

The endoscope apparatus may include an autofocus section that performs an autofocus process, and an observation distance acquisition section that acquires observation distance information about a distance between the endoscope apparatus and the object based on the autofocus process. The observation magnification acquisition section 209 may acquire the magnification state information based on the observation distance information.

The observation magnification acquisition section 209 may thus acquire information based on the autofocus process as the magnification state information. Specifically, the observation magnification acquisition section 209 may acquire the observation distance information about the distance between the endoscope apparatus and the object as a result of the autofocus process. More specifically, the observation magnification acquisition section 209 may set the observation mode to the magnifying observation mode (i.e., a mode in which the blurring correction process is performed) when the observation distance is short (i.e., the object is observed in detail), and may set the observation mode to the normal observation mode (i.e., a mode in which the blurring correction process is not performed) when the observation distance is long (i.e., a screening process is performed).

The light source control section 206 may include an aperture control section, and may control the intensity of illumination light based on aperture control performed by the aperture control section.

This makes it possible to control the intensity of illumination light based on the state (open/close) of the aperture of the optical system. Therefore, it is possible to deal with a shortage of the intensity of illumination light due to a reduction in the imaging period by opening the aperture up using the aperture control section.

The light source control section 206 may include a current control section that controls a current that flows through a light source, and may control the intensity of illumination light based on the current controlled by the current control section.

This makes it possible to control the intensity of illumination light based on the current that flows through the light source (e.g., LED light source), for example. Therefore, it is possible to deal with a shortage of the intensity of illumination light due to a reduction in the imaging period by increasing the amount of current that flows through the light source using the current control section.

The first embodiment also relates to a method of controlling an endoscope apparatus that includes acquiring the magnification state information, reducing the imaging period based on the acquired magnification state information, and controlling the intensity of illumination light corresponding to the reduced imaging period.

This makes it possible to achieve the above effects by applying the method according to the first embodiment to a control method instead of an endoscope apparatus.

### 3. Second Embodiment

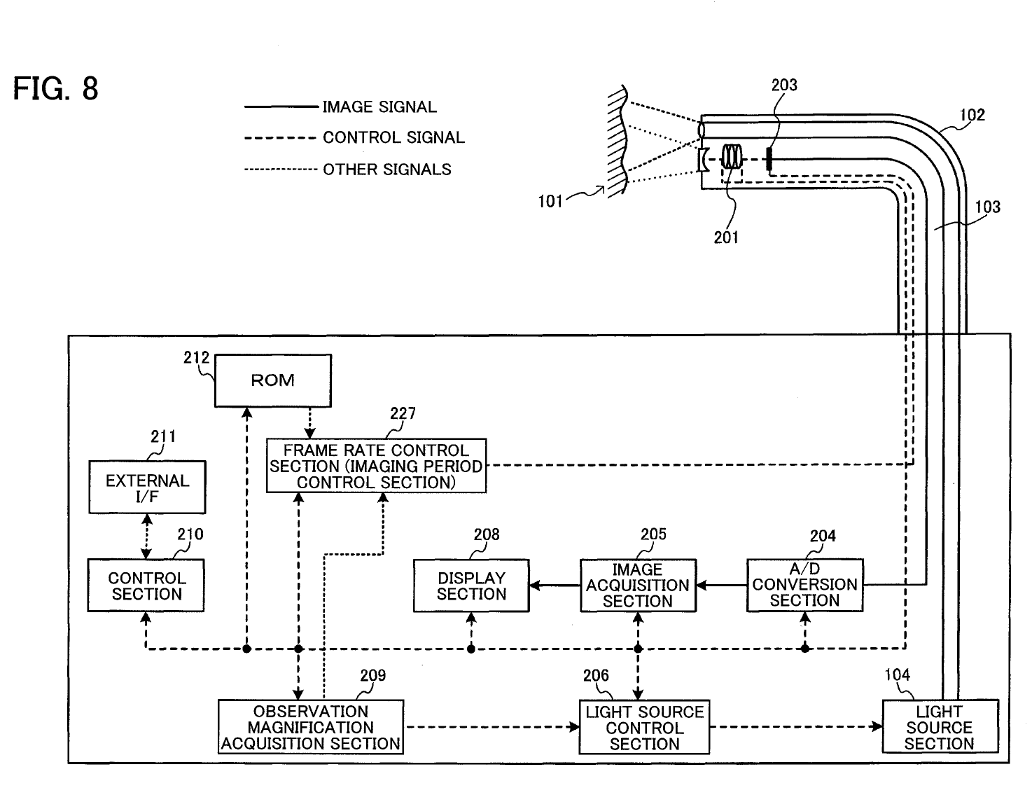

FIG. 8 shows a configuration example of an endoscope apparatus according to the second embodiment. The endoscope apparatus that observes an object 101 includes an insertion section 102, a light guide 103, a light source section 104, a lens system 201, an imaging element 203, an A/D conversion section 204, an image acquisition section 205, a light source control section 206, a frame rate control section 227, a display section 208, an observation magnification acquisition section 209, a control section 210, an external I/F 211, and a ROM 212.

The endoscope apparatus according to the second embodiment is configured in the same manner as the endoscope apparatus according to the first embodiment, except that the frame rate control section 227 is provided instead of the shutter control section 207. Detailed description of a configuration similar to that of the endoscope apparatus according to the first embodiment is omitted.

The endoscope apparatus according to the second embodiment is configured so that blurring of the image is suppressed by increasing the frame rate, and the brightness of the image signal is maintained by increasing the intensity of light emitted from the light source during magnifying observation.

Specifically, when the user has moved the end of the endoscope apparatus closer to the lesion area, the control section 210 adjusts the lens system 201 based on operation information input from the external I/F 211 so that the observation mode is changed to magnifying observation. The observation magnification acquisition section 209 acquires observation magnification information (or information that directly changes the observation mode to magnifying observation or normal observation) from the control section 210, and causes the light source control section 206 and the frame rate control section 227 to operate. The frame rate control section 227 adjusts the imaging period by controlling the frame rate.

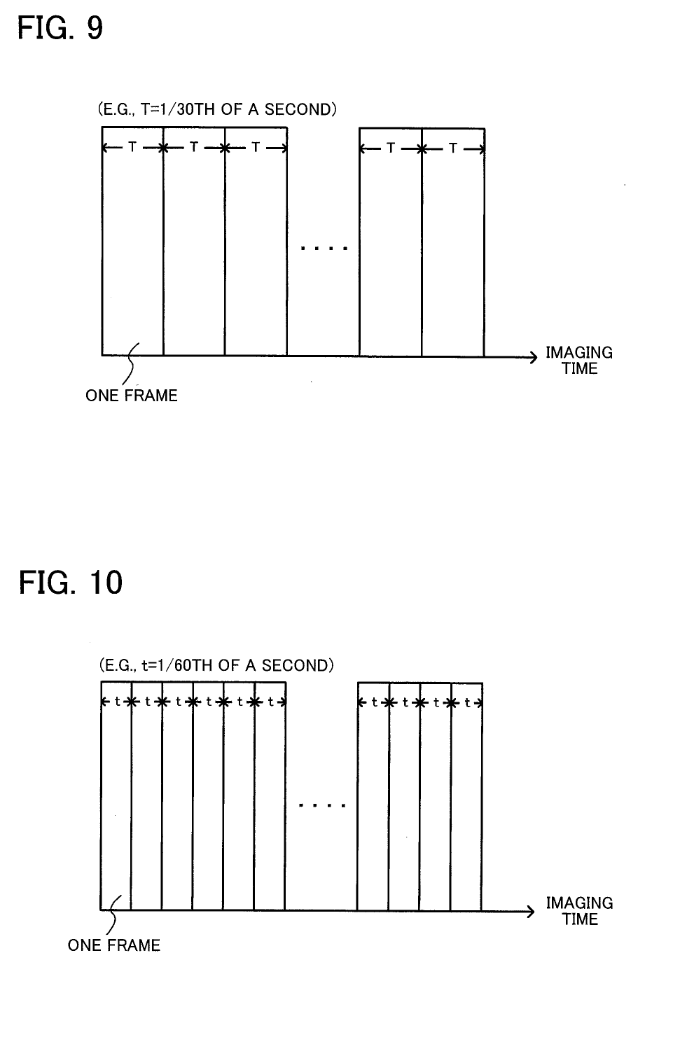

For example, when the frame rate during normal observation is 30 fps (see FIG. 9), the frame rate is set to 60 fps during magnifying observation (see FIG. 10). Therefore, the exposure time of the imaging element per frame decreases (e.g., decreases to 1/60th of a second from 1/30th of a second in the above example), so that the effects of blurring can be reduced. In this case, it is necessary to increase the intensity of illumination light since the image darkens. The intensity of illumination light is increased in the same manner as in the first embodiment (detailed description thereof is omitted).

A normal endoscope dimming process that does not utilize the method according to the second embodiment is characterized in that the brightness of the image signal is measured in each observation state, and the intensity of light emitted from the light source and the gain of the image signal are gradually adjusted so that the image does not suddenly become dark or bright. Therefore, when the frame rate is increased by a factor of 4, for example, the image signal temporarily becomes dark, and then gradually becomes bright. This may be stressful for the user. On the other hand, the method according to the second embodiment increases the frame rate during magnifying observation, and instantaneously increases the intensity of light emitted from the light source corresponding to the frame rate, so that blurring of the image can be suppressed while maintaining the brightness of the image signal.

The imaging period control section according to the second embodiment includes the frame rate control section 227 that adjusts the frame rate corresponding to the magnification state information (see FIG. 8), and controls the imaging period corresponding to the frame rate adjusted by the frame rate control section 227.

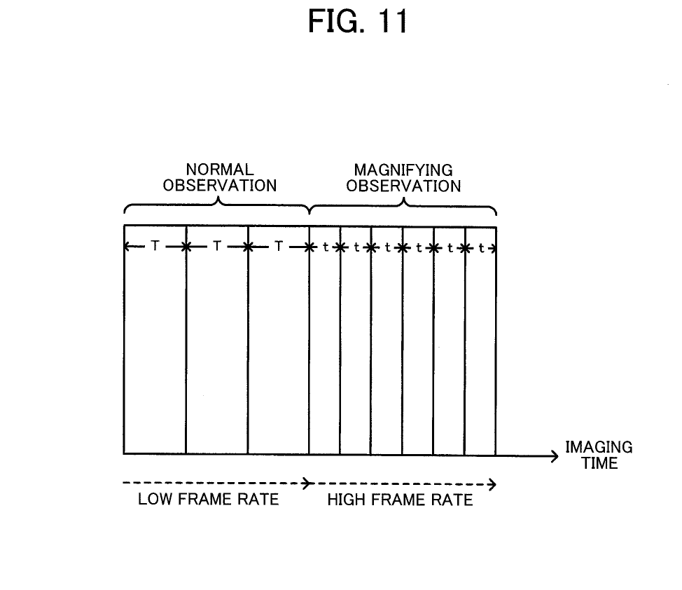

Therefore, the imaging period control section can control the imaging period based on the frame rate (see FIG. 11).

### 4. Third Embodiment

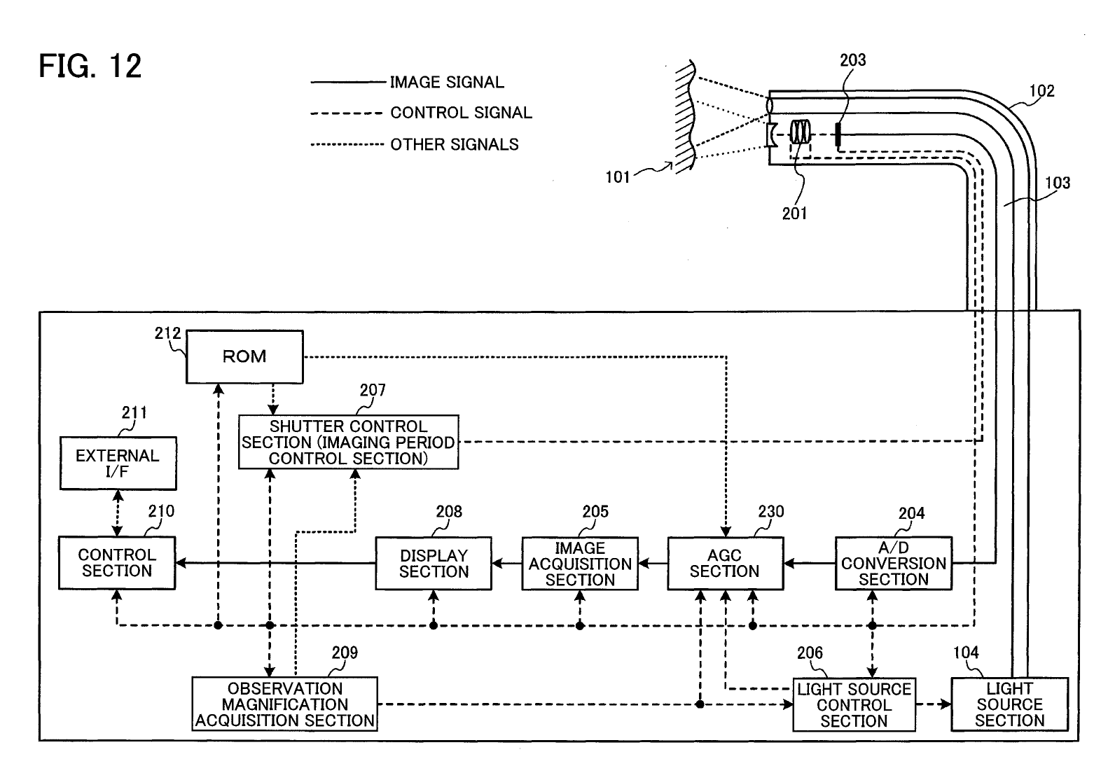

FIG. 12 shows a configuration example of an endoscope apparatus according to the third embodiment. The endoscope apparatus that observes an object 101 includes an insertion section 102, a light guide 103, a light source section 104, a lens system 201, an imaging element 203, an A/D conversion section 204, an AGC section 230, an image acquisition section 205, a light source control section 206, a shutter control section 207, a display section 208, an observation magnification acquisition section 209, a control section 210, an external I/F 211, and a ROM 212.

Note that the following description focuses on the differences from the first embodiment.

The A/D conversion section 204 is connected to the display section 208 via the AGC section 230 and the image acquisition section 205. The observation magnification acquisition section 209 is connected to the AGC section 230, the light source control section 206, and the shutter control section 207. The shutter control section 207 is connected to the imaging element 203. The ROM 212 is connected to the shutter control section 207 and the AGC section 230. The light source control section 206 is connected to the light source section 104 and the AGC section 230. The control section 210 is bidirectionally connected to the lens system 201, the A/D conversion section 204, the AGC section 230, the image acquisition section 205, the light source control section 206, the shutter control section 207, the observation magnification acquisition section 209, the external I/F 211, and the ROM 212.

The endoscope apparatus according to the third embodiment is configured so that the light source aperture 302 is opened up (i.e., the intensity of light emitted from the light source is increased) during magnifying observation under control of the light source control section 206 in the same manner as in the first embodiment. However, the brightness of the image signal may not be sufficiently corrected due to physical limitations even if the light source aperture 302 is opened up to a maximum extent (e.g., even if the amount of current that flows through an LED is maximized). The above problem may significantly occur when observing the object while applying special light within a narrow wavelength band (e.g., NBI or AFI).

In order to solve the above problem, the endoscope apparatus according to the third embodiment is configured so that the AGC section 230 performs a gain control process under control of the control section 210 when it has been determined that the brightness of the image signal cannot be sufficiently corrected even if the light source aperture 302 is opened up to a maximum extent, and corrects (compensates for) the shortage of the brightness of the image signal after the light source has been controlled.

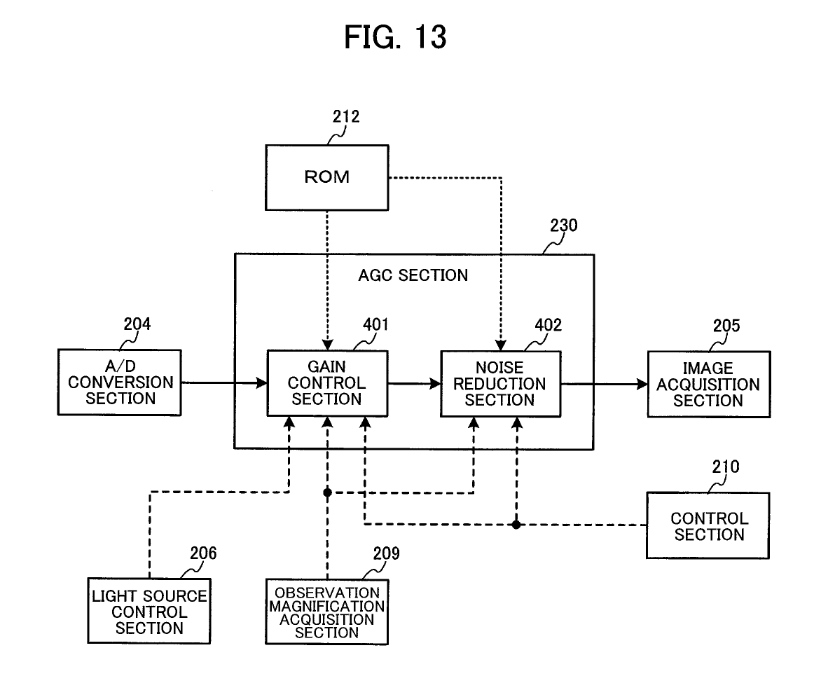

FIG. 13 shows an example of the configuration of the AGC section 230. The AGC section 230 includes a gain control section 401 and a noise reduction section 402. The A/D conversion section 204 is connected to the image acquisition section 205 via the gain control section 401 and the noise reduction section 402. The ROM 212 is connected to the gain control section 401 and the noise reduction section 402. The observation magnification acquisition section 209 is connected to the gain control section 401 and the noise reduction section 402. The light source control section 206 is connected to the gain control section 401. The control section 210 is bidirectionally connected to the gain control section 401 and the noise reduction section 402.

The gain control section 401 extracts a gain value that adjusts the brightness of the image signal corresponding to the observation state from the ROM 212 based on observation state information input from the observation magnification acquisition section 209 (information that indicates normal observation or magnifying observation) and information about a change in the intensity of illumination light emitted from the light source during magnifying observation input from the light source control section 206, and multiplies an image signal value input from the A/D conversion section 204 by the gain value.

For example, when the shutter speed during magnifying observation is 1/120th of a second, and the shutter speed during normal observation is 1/30th of a second, the exposure time during magnifying observation is a quarter of the exposure time during normal observation, and the brightness of the image signal acquired during magnifying observation is also a quarter of the brightness of the image signal acquired during normal observation. In this case, the brightness of the image signal can be maintained by increasing the intensity of illumination light emitted from the light source by a factor of 4. For example, when it has been determined that the intensity of illumination light can only be doubled as compared with the intensity of illumination light before the shutter speed is adjusted, even if the light source aperture 302 is opened up to a maximum extent, the light source control section 206 doubles the intensity of illumination light by opening up the light source aperture 302 to a maximum extent, and transmits information that indicates to that effect to the gain control section 401. The gain control section 401 determines that the brightness of the image signal during magnifying observation obtained by multiplying the image signal value by the gain value is equal to the brightness of the image signal during normal observation by doubling the gain value that is multiplied by the image signal during magnifying observation input from the A/D conversion section 204 as compared with the gain value during normal observation. The gain control section 401 then transmits the image signal multiplied by the gain value to the noise reduction section 402 under control of the control section 210.

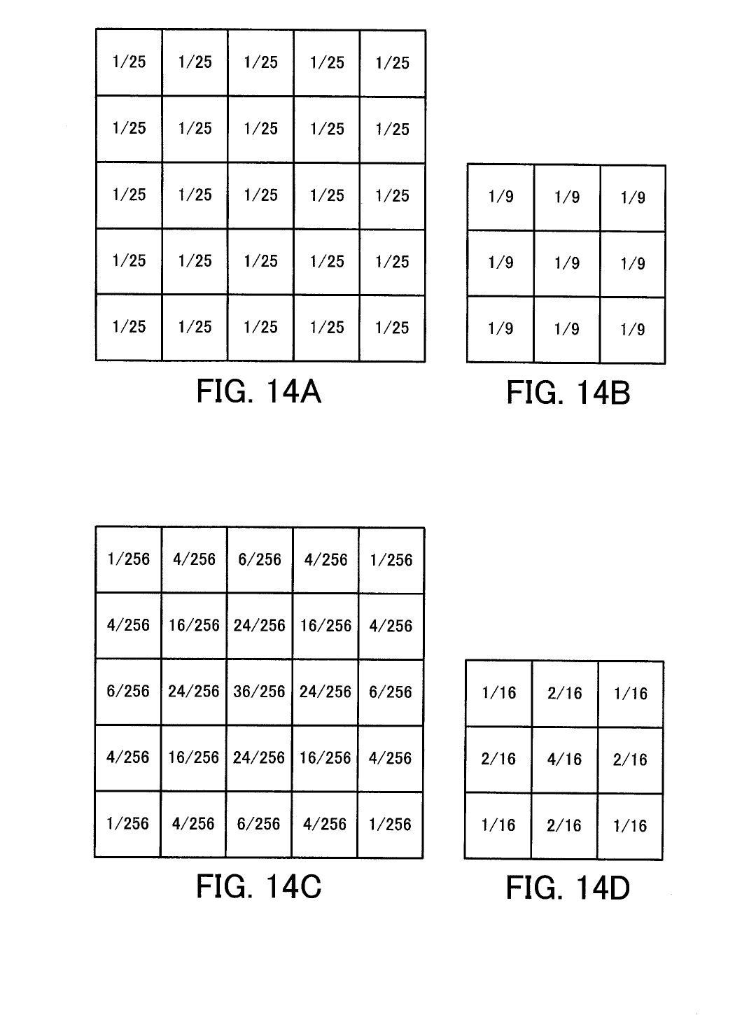

However, when the gain value is doubled to maintain the brightness of the image signal during magnifying observation, noise contained in the image signal value multiplied by the gain value is amplified by a factor of 2. In order to solve the above problem, the noise reduction section 402 extracts a noise-reduction smoothing filter coefficient corresponding to imaging state information input from the ROM 212 based on the observation state information (normal observation or magnifying observation) input from the observation magnification acquisition section 209, and performs a noise reduction process. As shown in FIGS. 14A to 14D, the noise reduction section 402 performs a smoothing process on each pixel indicated by the image signal acquired during normal observation using a filter B (or D), for example. On the other hand, the noise reduction section 402 performs the smoothing process on each pixel indicated by the image signal acquired during magnifying observation using a filter A (or C). Since a 5×5 filter has an increased smoothing effect as compared with a 3×3 filter, the effect of suppressing noise components amplified by the gain multiplication process increases.

The noise reduction section 402 transmits the image signal subjected to the smoothing process to the image acquisition section 205. Note that the noise reduction section 402 may not perform the smoothing process during normal observation.

As described above, the shutter speed is increased during magnifying observation, and the intensity of illumination light is increased by opening up the light source aperture 302. The image signal is corrected by the AGC process or the like so that the brightness of the image signal does not decrease when the intensity of illumination light is insufficient even if the light source aperture 302 is opened up to a maximum extent. Since the noise-reduction coefficient can be controlled by the noise reduction process, and noise components contained in the image signal and amplified by the AGC process can be reduced, an image quality equal to that obtained during normal observation can be obtained while suppressing blurring of the image.

Although the third embodiment has been described above taking an example in which the shutter speed is increased in the same manner as in the first embodiment, the configuration according to the third embodiment is not limited thereto. For example, the frame rate may be increased in the same manner as in the second embodiment, or the frame rate and the shutter speed may be increased at the same time.

The endoscope apparatus according to the third embodiment includes the gain control section 401 that performs the gain control process on the signal value of the image signal, and the noise reduction section 402 that performs the noise reduction process on the image signal subjected to the gain control process (see FIG. 13).

This makes it possible to perform the gain-up process and the noise reduction process on the image that has darkened due to the blurring correction process. Therefore, it is possible to obtain a bright image with a small amount of noise as compared with the case where the gain-up process and the noise reduction process are not performed.

The gain control section 401 may perform the gain-up process using a gain value that brings the brightness of the image signal closer to a given reference value when the brightness of the image signal acquired in a state in which the intensity of illumination light is maximized by the light source control section 206 is lower than the given reference value. The noise reduction section 402 may adjust the noise reduction strength based on the gain value used for the gain-up process, and perform the noise reduction process.

This makes it possible to acquire a bright image signal when performing the blurring correction process by initially increasing the intensity of light using the light source control section 206, and then performing the gain-up process by image processing when the brightness of the image signal is insufficient (i.e., when the brightness of the image signal does not reach a given reference value even if the intensity of light is maximized). Therefore, it is possible to obtain an image with a small amount of noise by giving priority to the light intensity increase process performed by the light source control section 206 over the gain-up process that increases noise. Moreover, noise can be efficiently reduced by performing the noise reduction process subsequent to the gain-up process based on the gain value.

### 5. Fourth Embodiment

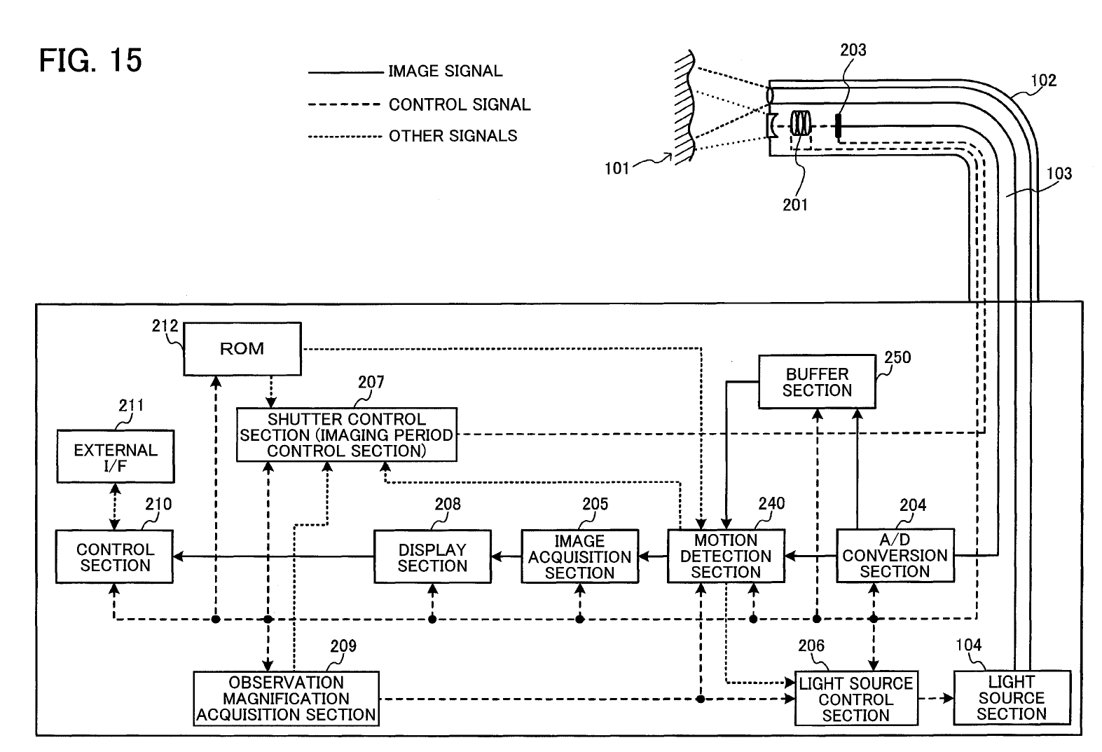

FIG. 15 shows a configuration example of an endoscope apparatus according to the fourth embodiment. The endoscope apparatus that observes an object 101 includes an insertion section 102, a light guide 103, a light source section 104, a lens system 201, an imaging element 203, an A/D conversion section 204, a motion detection section 240, a buffer section 250, an image acquisition section 205, a light source control section 206, a shutter control section 207, a display section 208, an observation magnification acquisition section 209, a control section 210, an external I/F 211, and a ROM 212.

Note that the following description focuses on the differences from the first to third embodiments.

The A/D conversion section 204 is connected to the display section 208 via the motion detection section 240 and the image acquisition section 205. The observation magnification acquisition section 209 is connected to the motion detection section 240, the light source control section 206, and the shutter control section 207. The A/D conversion section 204 is connected to the buffer section 250. The ROM 212 is connected to the shutter control section 207 and the motion detection section 240. The motion detection section 240 is connected to the light source control section 206 and the shutter control section 207. The control section 210 is bidirectionally connected to the lens system 201, the A/D conversion section 204, the motion detection section 240, the buffer section 250, the image acquisition section 205, the light source control section 206, the shutter control section 207, the observation magnification acquisition section 209, the external I/F 211, and the ROM 212.

The A/D conversion section 204 converts the analog image signal output from the imaging element 203 into a digital image signal (hereinafter referred to as “image signal”), and transmits the image signal to the image acquisition section 205 and the buffer section 250. The buffer section 250 stores the image signal in the current frame and the image signal in the preceding frame in time series. When the buffer section 250 has received a new image signal (image signal in a new frame), the buffer section 250 updates the old image signal in time series with the new image signal.

The motion detection section 240 determines the observation state from observation state information (information that indicates magnifying observation or normal observation based on observation magnification information) input from the observation magnification acquisition section 209. When the observation state information indicates magnifying observation, the motion detection section 240 performs a process that divides an image into a plurality of block areas having a given size on the image signal in the current frame input from the A/D conversion section 204 and the image signal in the preceding frame input from the buffer section 250, and performs a motion detection process by performing a known template matching process.

The motion detection section 240 calculates a global motion amount in the current frame by calculating a weighted average of the motion amount in each block area. The motion detection section 240 compares the calculated global motion amount with a given motion amount threshold value. The motion detection section 240 determines that the motion amount is large when the global motion amount is larger than the motion amount threshold value, and transmits motion determination information to the shutter control section 207 and the light source control section 206.

The light source control section 206 determines whether or not to adjust the intensity of illumination light based on the observation state information input from the observation magnification acquisition section 209 and the motion determination information input from the motion detection section 240. When the light source control section 206 has determined that the observation state is magnifying observation and the motion amount is large, the light source control section 206 increases the intensity of illumination light emitted from the light source section 104. The shutter control section 207 determines whether or not to adjust the shutter speed based on the observation state information input from the observation magnification acquisition section 209 and the motion determination information input from the motion detection section 240. When the shutter control section 207 has determined that the observation state is magnifying observation and the motion amount is large, the shutter control section 207 increases the shutter speed by adjusting the exposure time by controlling the electronic shutter of the imaging element.

Blurring of the image can be suppressed while maintaining the brightness of the image signal by detecting the motion amount during magnifying observation, and increasing the shutter speed and the intensity of illumination light when it has been determined that the motion amount is large.

Although the fourth embodiment has been described above taking an example in which the shutter speed is increased in the same manner as in the first embodiment, the configuration according to the fourth embodiment is not limited thereto. For example, the frame rate may be increased in the same manner as in the second embodiment, or the frame rate and the shutter speed may be increased at the same time.

As shown in FIG. 15, the endoscope apparatus according to the fourth embodiment includes the motion detection section that detects motion amount information that indicates a relative motion amount of the object and the imaging section. The imaging period control section reduces the imaging period based on the motion amount information and the magnification state information. The imaging period control section may reduce the imaging period when it has been determined that the motion amount indicated by the motion amount information is larger than a given motion amount threshold value, and the observation magnification indicated by the magnification state information is higher than a given observation magnification threshold value.

This makes it possible to control the imaging period based on the magnification state information and the motion amount. The blurring correction process need not necessarily be performed during magnifying observation when a motion that results in blurring is small. According to the fourth embodiment, since the blurring correction process (imaging period control process) is performed when the observation state is magnifying observation and the motion amount is large, an unnecessary process can be omitted, so that the process can be simplified.

### 6. Fifth Embodiment

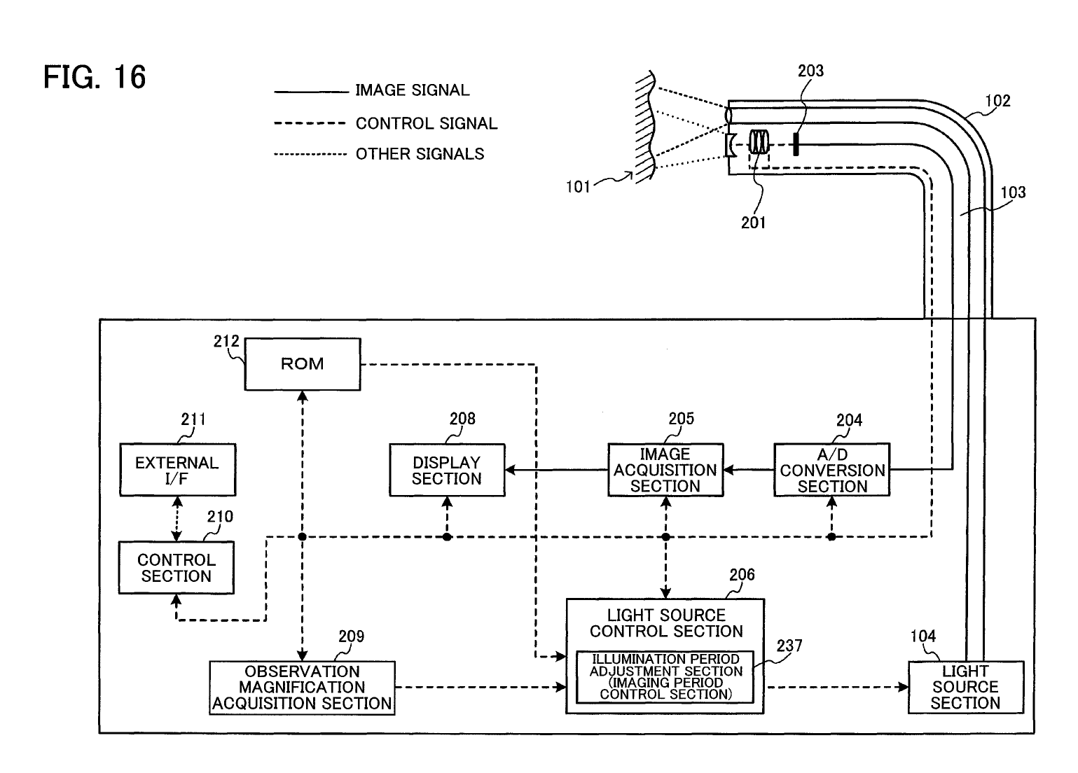

FIG. 16 shows an example of the configuration of an endoscope apparatus according to the fifth embodiment. The endoscope apparatus that observes an object 101 includes an insertion section 102, a light guide 103, a light source section 104, a lens system 201, an imaging element 203, an A/D conversion section 204, an image acquisition section 205, a light source control section 206 (illumination period adjustment section 237), a display section 208, an observation magnification acquisition section 209, a control section 210, an external I/F 211, and a ROM 212.

The endoscope apparatus according to the fifth embodiment is configured in the same manner as the endoscope apparatus according to the first embodiment, except that the shutter control section 207 is omitted, and the illumination period adjustment section 237 is included in the light source control section 206. Note that detailed description of the same configuration as that of the endoscope apparatus according to the first embodiment is omitted.

The endoscope apparatus according to the fifth embodiment is configured so that the brightness of the image signal is maintained during magnifying observation by increasing the intensity of light emitted from the light source while suppressing blurring of the image by reducing the illumination period using light emitted from the light source.

Specifically, when the user has moved the end of the endoscope apparatus closer to the lesion area, the control section 210 adjusts the lens system 201 based on operation information input from the external I/F 211 so that the observation mode is changed to magnifying observation. The observation magnification acquisition section 209 acquires observation magnification information (or information that directly changes the observation mode to magnifying observation or normal observation) from the control section 210, and causes the light source control section 206 and the illumination period adjustment section 237 included in the light source control section 206 to operate. The illumination period adjustment section 237 adjusts the imaging period by controlling the illumination period using illumination light.

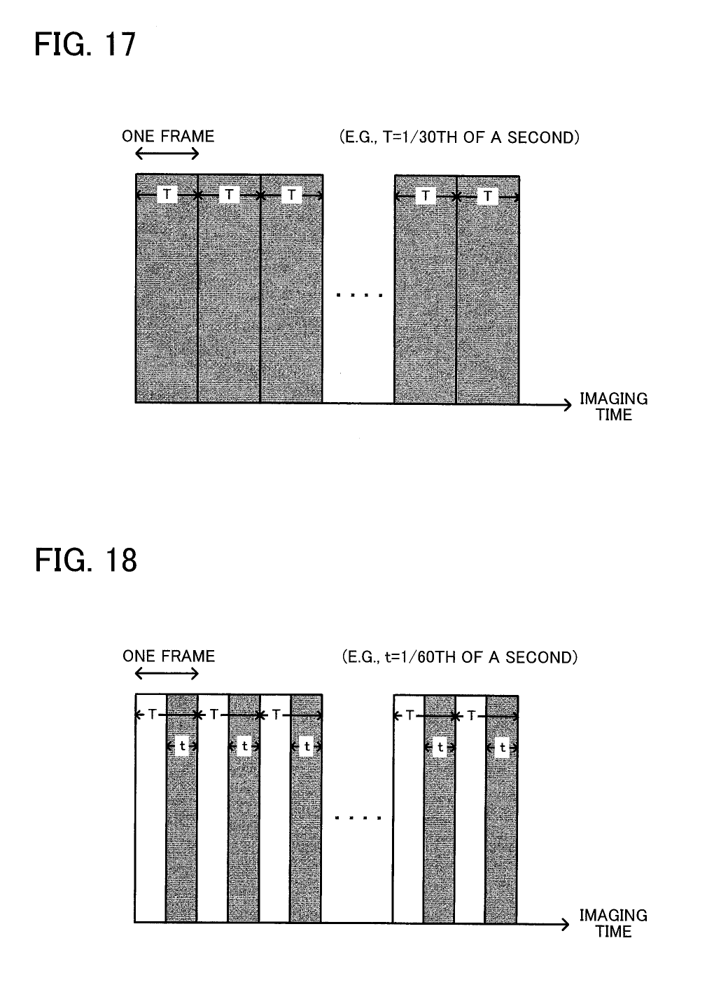

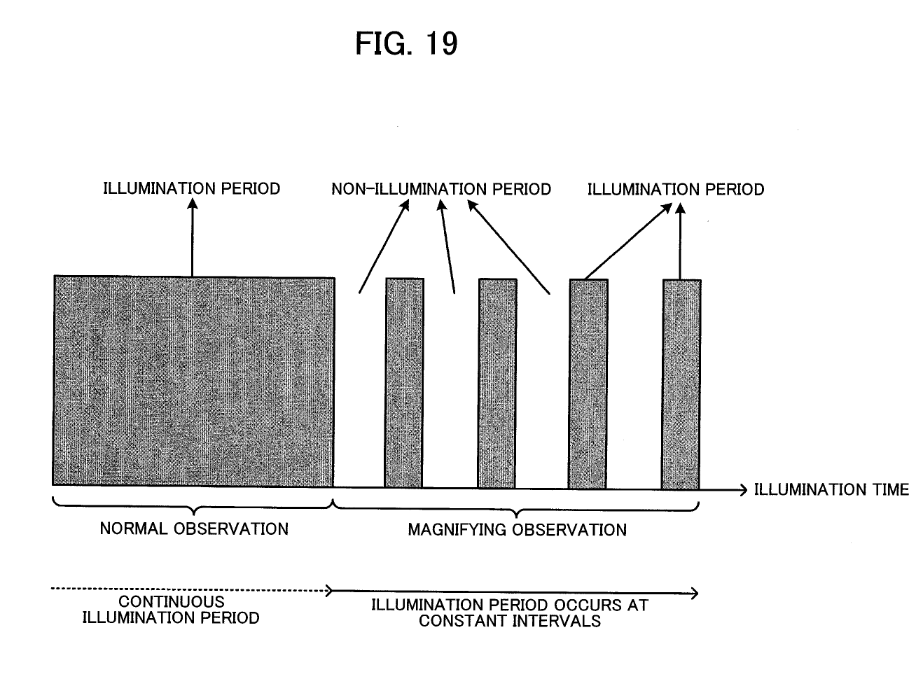

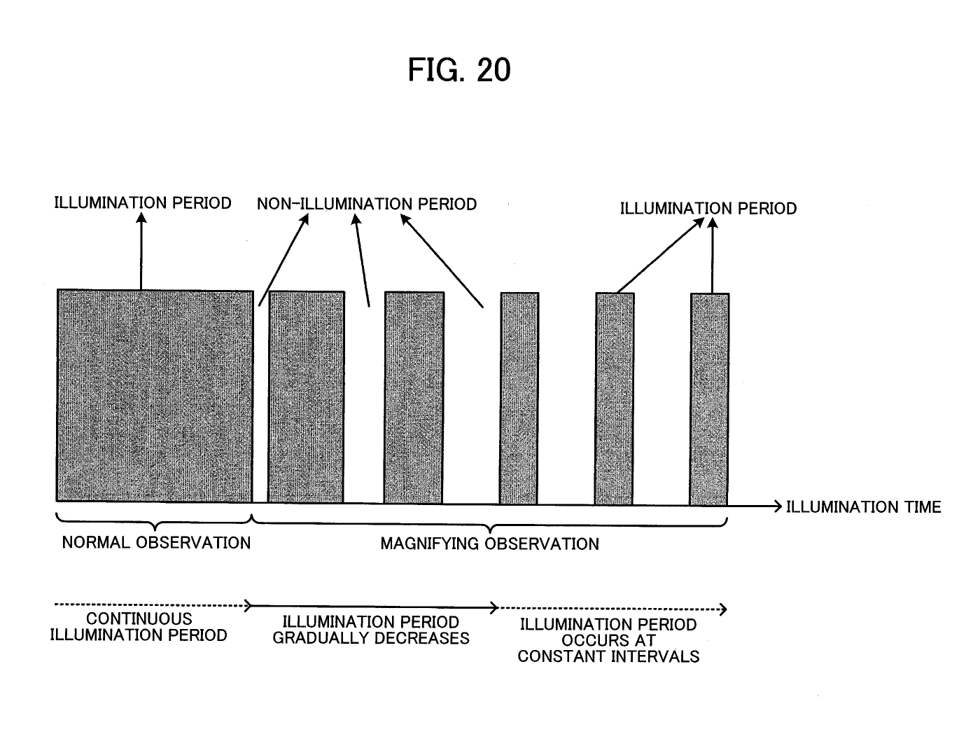

For example, when the illumination period during normal observation is 1/30th of a second (see FIG. 17), the illumination period is set to 1/60th of a second during magnifying observation (see FIG. 18). In FIGS. 17 and 18, a shaded area corresponds to the illumination period. This makes it possible to reduce the effects of blurring. In this case, it is necessary to increase the intensity of illumination light since the image darkens. The intensity of illumination light is increased in the same manner as in the first embodiment (detailed description thereof is omitted). The illumination period may be instantaneously reduced (see FIG. 19), or may be gradually reduced (see FIG. 20).

The difference between the first embodiment and the fifth embodiment is further described below. According to the first embodiment, the imaging period is reduced by opening or closing the electronic shutter up or down (i.e., changing the charge storage time) while always applying illumination light to the object. According to the fifth embodiment, the imaging period is reduced by reducing the illumination period (blinking light emitted from the light source) without changing the charge storage time. Note that the first embodiment may be modified so that illumination light is not always applied to the object, and the fifth embodiment may be modified so that the charge storage time is changed.

Blurring of the image can be suppressed while maintaining the brightness of the image signal by reducing the illumination period using light emitted from the light source, and increasing the intensity of light emitted from the light source during magnifying observation.

The imaging period control section according to the fifth embodiment includes the illumination period adjustment section 237 that adjusts the illumination period using light emitted from the light source corresponding to the magnification state information (see FIG. 16), and controls the imaging period corresponding to the illumination period adjusted by the illumination period adjustment section 237.

Therefore, the imaging period control section can control the imaging period based on the illumination period (see FIGS. 19 and 20). The invention requires the imaging period control process for blurring correction, and the light source control process for providing a sufficient light intensity. According to the fifth embodiment, the imaging period control process and the light source control process can be performed by the light source control section 206 (see FIG. 16). This makes it possible to simplify the configuration of the endoscope apparatus.

The first to fifth embodiments according to the invention and modifications thereof have been described above. Note that the invention is not limited to the first to fifth embodiments and modifications thereof. Various modifications and variations may be made without departing from the scope of the invention. A plurality of elements described in connection with the first to fifth embodiments and modifications thereof may be appropriately combined. For example, some elements may be omitted from the elements described in connection with the first to fifth embodiments and modifications thereof. Some of the elements described in connection with the first to fifth embodiments and modifications thereof may be appropriately combined. Specifically, various modifications and applications are possible without materially departing from the novel teachings and advantages of the invention.

What is claimed is:

1. An endoscope apparatus comprising:
an observation magnification acquisition section that acquires magnification state information that indicates a state of an observation magnification;
a motion detection section that detects motion amount information that indicates a relative motion amount of an object and an imaging section of the endoscope apparatus;
an imaging period control section that reduces an imaging period of each image that is indicated by an image signal captured by the endoscope apparatus based on the magnification state information and the motion amount information; and
a light source control section that controls intensity of illumination light applied to the object corresponding to the imaging period.

2. The endoscope apparatus as defined in claim 1,
the light source control section increasing the intensity of the illumination light when the imaging period control section has reduced the imaging period.

3. The endoscope apparatus as defined in claim 2, further comprising:
a gain control section that performs a gain control process on a signal value of the image signal; and
a noise reduction section that performs a noise reduction process on the image signal that has been subjected to the gain control process by the gain control section.

4. The endoscope apparatus as defined in claim 3,
the gain control section amplifying the signal value of the image signal using a gain value that brings brightness of the image signal closer to a given reference value when the brightness of the image signal acquired in a state in which the intensity of the illumination light is maximized by the light source control section is lower than the given reference value, and
the noise reduction section adjusting a strength of the noise reduction process performed on the image signal based on the gain value, and performing the noise reduction process on the image signal using the adjusted strength.

5. The endoscope apparatus as defined in claim 1,
the imaging period control section reducing the imaging period in a magnifying observation mode as compared with a normal observation mode, and
the light source control section increasing the intensity of the illumination light in the magnifying observation mode as compared with the normal observation mode.

6. The endoscope apparatus as defined in claim 1,
the observation magnification acquisition section acquiring adjustment information about the observation magnification that has been input by a user as the magnification state information, and
the imaging period control section reducing the imaging period based on the acquired magnification state information.

7. The endoscope apparatus as defined in claim 1,
the imaging period control section reducing the imaging period when it has been determined that the observation magnification indicated by the magnification state information is equal to or higher than a given threshold value.

8. The endoscope apparatus as defined in claim 1,
the imaging period control section reducing the imaging period when it has been determined that the motion amount indicated by the motion amount information detected by the motion detection section is larger than a given motion amount threshold value, and the observation magnification indicated by the magnification state information acquired by the observation magnification acquisition section is higher than a given observation magnification threshold value.

9. The endoscope apparatus as defined in claim 1,
the imaging period control section including a shutter control section that adjusts a shutter speed when capturing each image corresponding to the magnification state information, and controlling the imaging period corresponding to the shutter speed adjusted by the shutter control section.

10. The endoscope apparatus as defined in claim 9,
the shutter control section adjusting the shutter speed based on an exposure time of an imaging element when capturing each image, and
the imaging period control section controlling the imaging period corresponding to the shutter speed adjusted by the shutter control section based on the exposure time.

11. The endoscope apparatus as defined in claim 1,
the imaging period control section including a frame rate control section that adjusts a frame rate when capturing each image corresponding to the magnification state information, and controlling the imaging period corresponding to the frame rate adjusted by the frame rate control section.

12. The endoscope apparatus as defined in claim 1,
the imaging period control section including an illumination period adjustment section that adjusts an illumination period using the illumination light when capturing each image corresponding to the magnification state information, and controlling the imaging period corresponding to the illumination period adjusted by the illumination period adjustment section.

13. The endoscope apparatus as defined in claim 1, further comprising:
an autofocus section that performs an autofocus process; and
an observation distance acquisition section that acquires observation distance information about a distance between the endoscope apparatus and the object based on the autofocus process performed by the autofocus section,
the observation magnification acquisition section acquiring the magnification state information based on the observation distance information.

14. The endoscope apparatus as defined in claim 1,
the light source control section including an aperture control section that controls an aperture of an optical system, and controlling the intensity of the illumination light based on aperture control performed by the aperture control section.

15. The endoscope apparatus as defined in claim 1,
the light source control section including a current control section that controls a current that flows through a light source, and controlling the intensity of the illumination light based on the current controlled by the current control section.

16. A method of controlling an endoscope apparatus comprising:
acquiring magnification state information and motion amount information, the magnification state information indicating a state of an observation magnification, and the motion amount information indicating a relative motion amount of an object and an imaging section of the endoscope apparatus;
reducing an imaging period of each image that is indicated by an image signal captured by the endoscope apparatus based on the acquired magnification state information and the acquired motion amount information; and
controlling intensity of illumination light applied to the object corresponding to the reduced imaging period.
